(19) United States
(12) Patent Application Publication
(10) Pub. No.: US 2010/0130997 A1
(43) Pub. Date: May 27, 2010

(54) Blood sampling device

(75) Inventors: Richard W. LeVaughn, Newnan, GA (US); Gwenn E. Kennedy, Ellenwood, GA (US); Christopher J. Ruf, Marietta, GA (US); Mitchell A. Solis, Cumming, GA (US); Avi M. Robbins, Longwood, FL (US); Jason R. Heath, Marietta, GA (US); Stephen J. Flynn, Peachtree City, GA (US); Stephanie J. Campbell, Kennesaw, GA (US); Carl E. Griffin, Marietta, GA (US); John C. Irwin, Woodstock, GA (US); Mary Kate Pynes, Dallas, GA (US)

Correspondence Address: GARDNER GROFF GREENWALD & VILLANUEVA. PC, 2018 POWERS FERRY ROAD, SUITE 800, ATLANTA, GA, 30339, US

(73) Assignee: FACET TECHNOLOGIES, LLC, MARIETTA, GA (US)

(21) Appl. No.: 10/569,021

(22) PCT Filed: Aug. 19, 2004
(86) PCT No.: PCT/US04/26981

§ 371 Date: Nov. 20, 2008

Related U.S. Application Data
(60) Provisional application No. 60/497,022, filed on Aug. 20, 2003.

(51) Int. Cl. A61B 17/32

(52) U.S. Cl. 606/172; 606/181; 606/182

(57) ABSTRACT

A lancing device for sampling blood or other body fluid of a subject includes a reusable housing and a disposable cassette. The cassette contains a plurality of lancets held in a storage plane. An active lancet is moved transversely from the storage plane into a firing plane, into engagement with a drive mechanism for actuation to lance the subject's skin. After actuation, the used lancet is returned to the storage plane. Each lancet includes a protective endcap that is removed from the lancet just prior to use and then is replaced onto the lancet after use.

### CROSS-REFERENCE TO RELATED APPLICATION

This application claims the benefit of U.S. Provisional Patent Application Ser. No. 60/497,022, filed Aug. 20, 2003, the entire content of which is hereby incorporated herein by reference for all purposes.

### FIELD OF THE INVENTION

The present invention relates generally to a medical sampling device, and more particularly to a multiple use device for minimally-invasive sampling of blood and/or other body fluids.

### BACKGROUND OF THE INVENTION

Many medical procedures require puncturing of the skin, and sometimes underlying tissues, of an animal or human subject. For example, a sharp lancet tip is commonly used to puncture the subject's skin at a lancing site to obtain a sample of blood, interstitial fluid or other body fluid, as for example in blood glucose monitoring by diabetics, and in blood typing and screening applications. In some instances, a person must periodically sample their blood for multiple testing throughout the day or week. Because re-use of a lancet can result in infection or spread of bloodborne contaminants, persons requiring repeated testing often must carry multiple lancets with them. This can be inconvenient and lead to reduced compliance with a prescribed test regimen. Accordingly, it can be seen that needs exist for a convenient, compact multi-use lancing device.

Various devices are known for sampling blood and other body fluids for analysis of the condition of a human or other animal subject. For example, U.S. Pat. No. 5,971,941 is understood to show a cassette with test strips for placement by a slider. A lancet pierces the skin surface so that blood can be obtained for analysis. The lancets are integrated on a test strip, and are positioned together with the test strip. Another embodiment is understood to show a disposable cylindrical insert having a lancet and a test membrane with an aperture for the lancet. The insert is inserted in a mounting cavity of a plunger or piston, which forces the lancet outward for blood withdrawal. DE 198 19 407 A1 is understood to show a multiplicity of test strips with integrated lancets for insertion into an analysis device.

U.S. Pat. No. 4,787,398 is understood to show a device with a plunger for directing a lancet outward, and has an evaluation system and a display system. A replaceable unit is applied to the device for each measurement. The replaceable unit comprises the lancet and a test strip, which is wetted with blood. This replaceable unit is thrown away after each use. EP 0 449 525 A1 is understood to show a blood withdrawal system wherein a new lancet is inserted manually into a release device before each use. A test strip is then inserted into the device. U.S. Pat. No. 4,627,445 is understood to show a device for measuring blood sugar, with an integrated blood withdrawal unit. A new replaceable lancet and test elements must be installed to the device for testing, and afterward disassembled. U.S. Pat. No. 5,951,492 is understood to show a disposable unit with a capillary tube and a test strip, to which sampled blood taken is applied. The capillary tube includes a lancet. A new disposable unit is attached and removed before and after each measurement.

EP 0877250 A2, EP 0949506 A2 and EP 811843 A2 are understood to show devices having a multiplicity of test elements arranged on a rotatable disk carrier. The test elements are brought successively into a working position and pushed out of the housing to be wetted with blood. U.S. Pat. No. 6,228,100 and U.S. Pat. No. 4,794,926 are understood to show lancets arranged on a carrier, which is rotated with respect to a housing.

German Application DE 100 57 832 C1 is understood to show a lancing device of a known form. Other lancing devices understood to include multiple lancets are shown, for example, in U.S. Patent App. Serial No. 2002/0087056 A1 and WO 02/36010 A1. EP 0589186 B1 is understood to show a lancet with a removable protective cap. WO 01/66010 A1 is understood to include a multiplicity of lancets in a magazine, with an opening of the chamber closed by an elastic material, which is penetrated in the puncture process.

Known sampling devices have, however, not proven fully satisfactory to all users for a variety of reasons. Accordingly, it is to the provision of an improved sampling device that the present invention is primarily directed.

### SUMMARY OF THE INVENTION

Briefly described, the present invention provides an improved sampling device, which is described herein by way of example embodiments representative of the various aspects of the invention. In one aspect, the invention is a lancing device having a plurality of penetration elements or lancets arranged for sequential use in piercing the skin or other tissue of a human or animal subject for obtaining a sample of blood, interstitial fluid, and/or other body fluid(s). The invention optionally can include collection and/or analysis features for collecting a sample of body fluid and/or analyzing one or more analytes or other characteristics of the sampled fluid. Certain embodiments are compact in size for convenience in portable personal use.

Example embodiments of the lancing device preferably include a housing and a multiplicity of lancets, with the multiplicity of lancets arranged on a carrier cartridge or cassette, which is insertable into the housing. A piston or plunger acts on a particular “active” or “working” lancet when oriented in its working position. The sharp point of the active lancet is driven into a skin surface of a user positioned over a lancing opening through the housing. The cassette is preferably removable from the housing after use for replacement.

In example embodiments, the invention comprises a single compact device contained within a housing. In the housing, a working position of the lancet corresponds to a puncture position to be applied to the skin surface of a user. Each of a plurality of lancets is moved sequentially into the working position in the device. When a lancet is positioned in its working position, the lancet is driven into the skin surface of a user.

Various embodiments of the invention provide an “all-in-one” device which is compact, i.e., is space-saving, and which is user-friendly and operator-friendly. Used lancets preferably are removed and replaced with unused lancets in a simple manner as part of a disposable replacement cassette or cartridge. In example embodiments, the lancets are arranged into or onto a carrier or cassette, which can be inserted into the lancing device and which can be rotated or otherwise moved with respect to the housing, such that the lancets can be moved into working positions, located adjacent or apart from each other, as for example by rotating the carrier or cassette within the housing. Therefore, the lancets are preferably arranged on a rotatable carrier and can be moved into the working position, as by a rotary movement. A lancet is in its working position if it can be moved out through the puncture position to puncture the skin.

Use of a rotatable carrier advantageously enables a compact design for the housing, such that used lancets are moved out of the working positions by rotating the carrier. In this way they come automatically to a disposal position without a need to provide another separate translational process. Optionally, lancets and test elements can be arranged on the same carrier, so that the lancets and test elements can, for example, be removed as a single manually operable unit from the packaging, and can be inserted into the analysis device in a single procedure. In other embodiments, the device includes lancets only, and serves as a lancing device without sampling and testing capability.

In still another embodiment of the invention, the lancets have removable caps at their free ends for protection against accidental sticks and to preserve sterility. The lancet tip can be forced through the protective capping means for sampling, or alternatively the protective capping means can be removed from the active lancet immediately before performing the puncture process. Cap removal can be accomplished in an advantageous manner by slightly retracting or drawing back the protective cap away from the active lancet immediately before performing the puncture process, and separating the cap from the path of movement of the lancet after it has been removed from the particular lancet, and holding it in a holding space.

In another aspect, the invention is a lancing device including a plurality of lancets positioned in a planar array, the planar array defining a storage plane, wherein an active lancet selected from the plurality of lancets is transversely displaced out of the storage plane into a firing plane.

In another aspect, the invention preferably comprises a lancing device having a reusable housing with an arming and firing mechanism, and a replaceable cassette. The cassette is removably mounted within the housing, and includes a plurality of lancets each having a lancet body and a protective cap. The cassette defines a lancet firing position, with the lancets being movable in turn into the firing position, and the lancets being held in a non-firing plane and selectively moved into a firing plane so that a lancet to be fired is in the firing plane while the other lancets are not in the firing plane. It is preferred that the lancets are moved into the firing plane by a cam as the lancets approach the firing position. It is also preferred that the lancets that have yet to be fired are held in the non-firing plane, are moved into the firing plane by a cam as the lancets approach the firing position, and then after firing are moved back into the non-firing plane but in a different position such that re-use of lancets is prevented.

Preferably, the lancets have an end shape that enables them to be captured positively and securely by the firing mechanism. In one form, this end shape is a T-shaped end structured to engage with and be captured by the arming and firing mechanism. In another form, this end is a ball-shaped end structured to engage with and be captured by the arming and firing mechanism.

Preferably, the lancets each include a removable cap, with the caps being pulled off the lancets just prior to the lancets being engaged and captured by the arming and firing mechanism. Advantageously, the cassette can include a cam track and each of the caps can include a follower which rides in and cooperates with the cam track such that at a certain point as the cap moves along the cam track, the cap is pulled off the lancet.

Also, it is preferred that the lancets are held stationary while the lancet caps are pulled off therefrom. In this regard, the caps are pulled from the lancets, rather than the other way around. It is also preferred that the lancets are guided by the arming and firing mechanism during firing of the lancets, and not by the cassette. This may provide more accurate and repeatable guidance of the lancets. Inasmuch as guiding errors can lead to less than ideal translation of the lancet toward the skin, guiding errors should be minimized to minimize pain and discomfort associated with using such a device. This is so because if the sharp lancet tip enters the skin with any instability or is oriented at an angle as it punctures the skin, the pain and discomfort experienced by the use may be greatly increased.

In example embodiments, the lancing device is configured such that the housing includes a base and a cover, with the cover having an opening and a movable door for selectively covering and uncovering the opening. Also, the base can include biasing elements for biasing the cassette toward the cover, which can help position the cassette within the housing and/or can assist in the subsequent removal of the cassette from the housing. Moreover, the lancing device cassette can initially include a breakaway tab to assist the user in placing the cassette in the housing. The breakaway tab can be broken off once the cassette is loaded in the housing.

In still another aspect, the invention is a lancing device including at least one lancet having a sharp tip and a protective endcap covering said sharp tip. The lancing device preferably further includes a decapping mechanism for removing the endcap from the lancet without radial displacement of, the lancet.

In another aspect, the invention is a lancing device including a lancet movable between a retracted position and a lancing position. The lancet preferably includes a lancet body with a wing projecting outwardly therefrom. The lancing device preferably further includes a retraction spring separate from the lancet, the retraction spring contacting the outwardly projecting wing when the lancet is in the lancing position to bias the lancet toward the retracted position.

In another aspect, the invention is a lancing device including a plurality of lancets, an advancing mechanism for advancing each of the plurality of lancets sequentially into an active position, and a drive mechanism for propelling an active lancet selected from the plurality of lancets from a retracted position to a lancing position, wherein the advancing mechanism includes an arming lever that is actuated to advance the lancets and energize the drive mechanism.

In yet another aspect, the invention is a lancing device including a reusable housing and a cartridge containing a plurality of lancets, the cartridge being removably installed within the housing, and the lancing device further includes means for preventing reuse of a lancet.

In another aspect, the invention is a disposable replacement cassette comprising a plurality of lancets for penetrating the skin surface or other body portion of a human or animal subject to obtain samples of blood and/or other fluids.

In another aspect, the invention is a cassette for removable insertion within a lancing device. The cassette preferably includes a plurality of lancets positioned in a planar array, the planar array defining a storage plane, and wherein an active lancet selected from the plurality of lancets is transversely displaceable out of the storage plane into a firing plane within the cassette.

In yet another aspect, the invention is a cassette for removable insertion within a lancing device. The cassette preferably includes a lancet having a lancet body, a sharp tip projecting from the lancet body, and a protective endcap separably affixed over the sharp tip. The protective endcap preferably includes a follower in engagement with a cam track, the cam track defining a path for axially displacing the protective endcap from the lancet body as the lancet is laterally advanced within the cassette.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWING FIGURES

FIG. 1 is a perspective, exploded top view of a sampling device according to one example embodiment of the invention.

FIG. 2 is a perspective, exploded top view of a cassette of the sampling device of FIG. 1.

FIG. 3 is a detailed view of a single lancet of the cassette of FIG. 2.

FIG. 4A is a bottom plan view of the cassette of the sampling device of FIG. 2.

FIG. 4B is a bottom perspective view of a portion of the cassette of the sampling device of FIG. 3.

FIGS. 5-7 are top views of the cassette of the sampling device of FIG. 1, showing the uncapping of a lancet and the preparation and firing of the lancet.

FIG. 8 is a bottom view of the cassette of the sampling device and depicts an arming mechanism portion thereof.

FIG. 9 is a top, perspective view of the cassette portion of the sampling device.

FIG. 10 is a top, perspective view of the cassette portion of the sampling device as shown in FIG. 9, with some elements thereof removed to reveal a cam portion thereof for causing individual lancets to move into a firing plane.

FIGS. 11-17 are bottom views of the cassette portion of the sampling device of FIG. 1, showing the sequence of operation of an arming mechanism in preparation for firing of a lancet.

FIG. 18 is a top partial view of a sampling device according to a second example embodiment of the invention.

FIG. 19 is a bottom partial view of a portion of the sampling device of FIG. 1.

FIG. 20 is a side sectional view of the device of FIG. 19, taken along view lines A-A of FIG. 19.

FIG. 21 is a top partial view of a portion of the sampling device of FIG. 18.

FIG. 22 is a side sectional view of the device of FIG. 21, taken along view lines A-A of FIG. 21.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

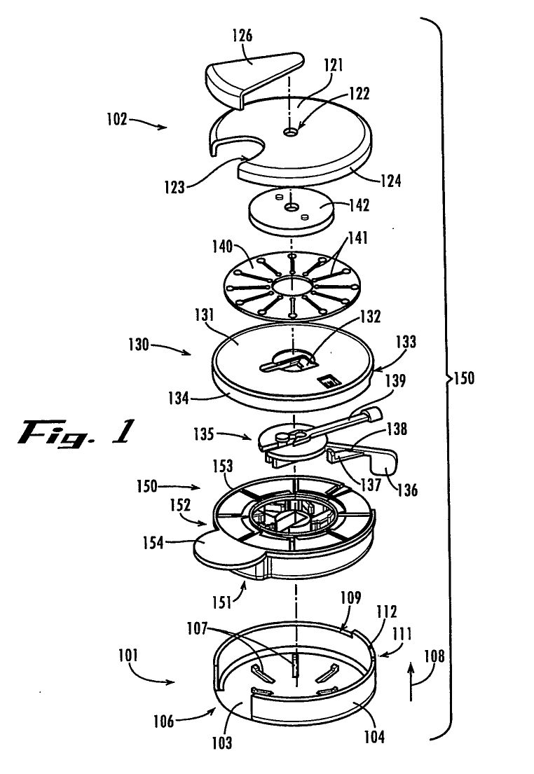

FIG. 1, as a perspective exploded view, shows a blood analysis device according to one example embodiment of the invention, which as a whole is designated by the reference number 100. The blood analysis device 100 comprises a housing base 101 and a housing cover 102, with the base and the cover collectively comprising a housing. The blood analysis device 100 also includes a cassette or cartridge 150, which contains a plurality of lancets and can optionally contain a plurality of test elements (not shown in this figure). Unused lancets (and, if present, test elements) are stored in the housing, used, and after use are discarded or disposed of by removing and replacing the entire cassette 150. The blood analysis device 100 can further comprise an analyte evaluation system, such as an electronic evaluation system and a display monitor, such as in the form of a visually readable display to show the result of an evaluation. The device can be an “all-in-one” device, comprising lancing features and sample collection and analysis and monitoring features. In alternate embodiments, the invention is a lancing device only, comprising the lancing features substantially as described, and omitting the sample collection and analysis features.

The housing base 101 of the blood analysis device 100 preferably comprises a generally dish-shaped shallow pan, having the general size, shape and appearance of a short cylinder. The housing base 101 includes a bottom 103 and a peripheral sidewall 104. An opening indicated generally at 106 is formed in the sidewall 104. Resilient fingers or springs, such as finger 107, are positioned on the interior of the bottom 103. These resilient fingers bias the cassette 150 upwardly in the direction of direction narrow 108. This biasing force assists in ensuring that the cassette 150 is properly positioned within the device 100 during use. The biasing force from the resilient fingers also assists in removing the cassette 150 for easy replacement.

The peripheral sidewall 104 has a top edge or rim 109 that is generally planar. However, a small landing or ledge indicated generally at 111 is positioned generally opposite, but not quite exactly opposite, the opening 106. The ledge 111 includes a smaller knob 112.

The upper cover portion 102 of the base is generally cylindrical and includes a top or roof panel 121 having a central small bore 122, a large opening or cutout section 123, and peripheral rim 124. A rotatable access door 126 is pivotally mounted to the cover 121 by way of a pin mounted in the small central bore 122. The access door 126 can be pivoted to selectively cover and uncover the cutout opening 123.

A drive mechanism 130 is preferably fitted within the underside of the cover 121. The drive mechanism 130 has an overall squat, cylindrical, plate-like shape, and includes an interior cover 131, an interlock system indicated generally at 132, and a slot 133 formed in a peripheral rim portion 134 of the interior cover 131. An arming mechanism indicated generally at 135 is fitted in the underside of the drive mechanism 130. The arming mechanism includes an arming lever 136 and a rotatable pawl 137. The rotatable pawl 137 is generally L-shaped and pivots about a small pivot pin 138. A piston 139 is provided for driving a lancet to be fired under the influence of a drive spring or other transport mechanism.

Optionally, an analyte sampling platen 140 is provided above the interior cover 131 and includes a number of chemical leads, such as chemical lead 141, for chemically or electrically communicating with a test element. A washer or spacer 142 is positioned above the platen 141 and beneath the exterior cover 102.

The cassette or cartridge 150 is generally disk-shaped, in the form of a short, squat cylinder, having a small projection indicated generally at 151 that is adapted to be fitted within the opening 106 of the base and to extend therethrough. This provides a positive means of accurately positioning the cassette 150 within the housing base 101 and ensuring a reliable, repeatable angular alignment therewith. To assist the user in placing the cassette 150 in the housing base 101, a breakaway handle 152 is provided. The breakaway handle 152 includes a generally C-shaped peripheral rim 153 and an outwardly extending tab or tongue portion 154. Once the cassette 150 is successfully placed within the housing of the device 100, the breakaway tab 154 can be removed. Removing the breakaway tab results in a more compact unit and also makes it more difficult to accidentally reuse the cassette, so as to minimize risk of contamination.

Referring now to FIG. 2, the cassette 150 is considered in more detail. The cassette 150 includes a dish or bottom 156 that includes a floor 157 and an upstanding peripheral wall 158. As mentioned previously, there is a bump-out section 151 extending beyond the otherwise circular peripheral shape of the bottom 156. An opening or aperture 161 is formed in this bump-out section, through which the tip of the active lancet passes upon actuation to lance the skin of a subject. A generally bell-shaped cam 162 projects upwardly from the floor 157. A central column 163 is positioned in the middle of the floor 157 and includes a slot 164 that is aligned with the opening or aperture 161.

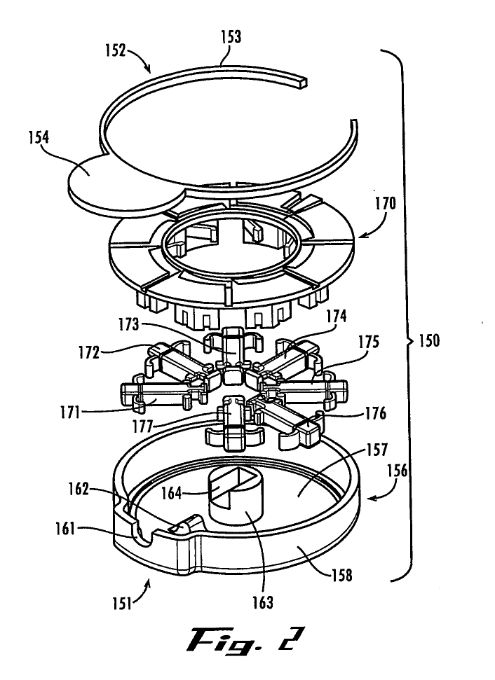

A carousel or carrier 170 is preferably positioned within the bottom 156 and has a plurality of lancets 171-177 mounted thereon. Each of the lancets preferably includes three main components: a lancet body, and needle or sharp secured within the lancet body, and a removable cap that covers the tip of the sharp. Moreover, as shown in FIG. 3, each lancet, indicated here at 180, has a unique external geometry and a number of novel features. The lancet preferably includes a resilient lancet body 181, a metal sharp 182 rigidly secured within the body 181, and a removable cap 183 for concealing the tip of the sharp and maintaining sterility. The cap 183 includes retaining arms or cantilevers 184, 186 extending laterally from side portions thereof, which arms preferably terminate in cylindrical ends 188, 189 for cooperative engagement with the carousel to control the position of the caps. In alternate embodiments, the arms 184, 186 are integrally formed with the carousel or are permanently affixed thereto. The cap 183 also includes a cam track follower 191, in the form of a short button, for following in a cam track, discussed below, to move the cap away from and back to the lancet body 181 as needed.

The lancet body 181 has a generally T-shaped end indicated generally at 192 and laterally extending wings 193, 194. The T-shaped end 192 cooperates with a corresponding channel or receiver opening of the drive piston to be captured therein for movement therewith during firing. The wings 193, 194 cooperate with unshown guide elements to guide the up and down movement of the lancet body relative to the remainder of the cassette, as for when the lancet body is moved into the firing plane and then back to the storage plane.

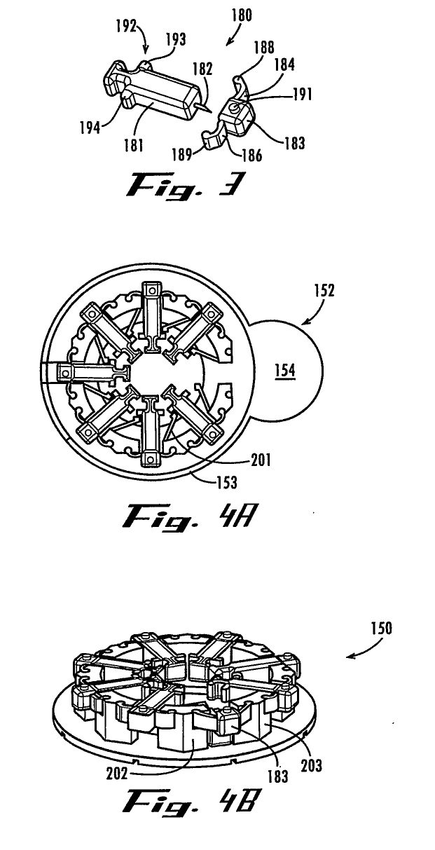

Referring now to FIG. 4A, the cassette 150 can be seen from the bottom. Each of the lancets includes a lancet retraction spring, such as retraction spring 201. These springs work to return the lancet to a position fully within the housing after firing. Advantageously, the retraction springs are not formed as part of the lancets, thereby reducing the weight and improving the dynamic balance of the lancets. Referring now to FIG. 4B, the cassette 150 can be seen from the bottom in perspective. The cassette 150 includes a number of guide blocks, such as guide blocks 202, 203, which help to control the position of the caps, such as protective cap 183. In this regard, the cantilevers or arms 184, 186 of the caps are retained in cylindrical bores formed in the guide blocks. In use, all of the caps, save one (the cap of the active lancet), are held fast against their respective lancet body. Thus, all lancets except the active lancet remain capped. Likewise, all but one of the lancets are held in a storage plane spaced from the base of the cassette 150. The uncapped active lancet is displaced transversely out of the storage plane and into the firing plane, preferably toward the base of the cassette 150, while the endcap of the active lancet remains in the storage plane. The arms 184, 186 extending from the lancet endcaps are preferably extensible, by means of a curved or angled profile or an elastic material of construction, so that their ends 188, 189 remain engaged with the guide blocks as the endcap is pulled away from the active lancet prior to advancement into engagement with the drive mechanism.

FIGS. 5-7 depict a preferred sequence of operation as the cartridge is rotationally advanced within the housing to move a lancet into and out of the active or firing position. As shown in FIG. 5, as lancet 171 approaches the firing position, its cap 183 is removed by the interplay of the cam track follower 191 on the lancet cap with the cam track 211. As can be appreciated from this figure, the cam track 211 is “bumped out” in the vicinity of region 151, which causes the cap to be pulled off of the relatively stationary lancet body as they both rotate in the counterclockwise direction indicated by direction arrow 212.

As shown in FIG. 6, as the lancet 171 moves into the active or firing position, with its cap 183 removed, the lancet is moved transversely, out of the storage plane and into the firing plane, by the engagement of the lancet against the bell-shaped cam 162. As a result of this movement, the T-shaped end of the lancet body is captured in the correspondingly shaped opening in the piston 139 of the drive mechanism, and is ready to be cocked and fired. This is accomplished as the cassette 150 (and the lancet body) is rotationally advanced in the counterclockwise direction of direction arrow 212.

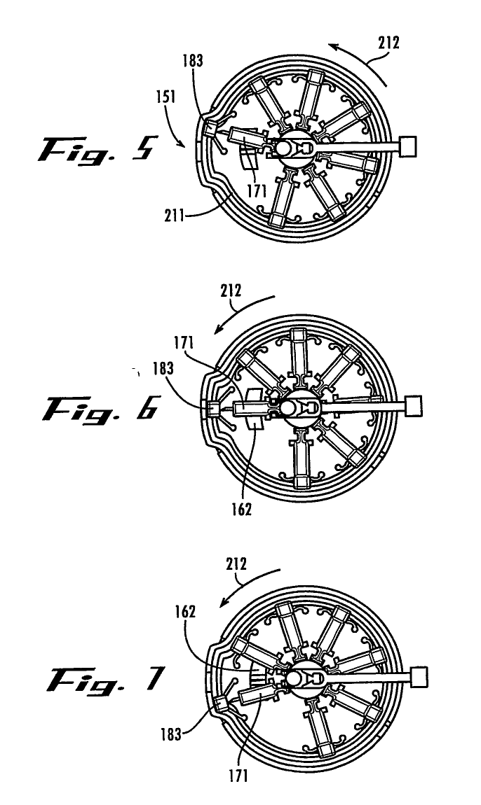

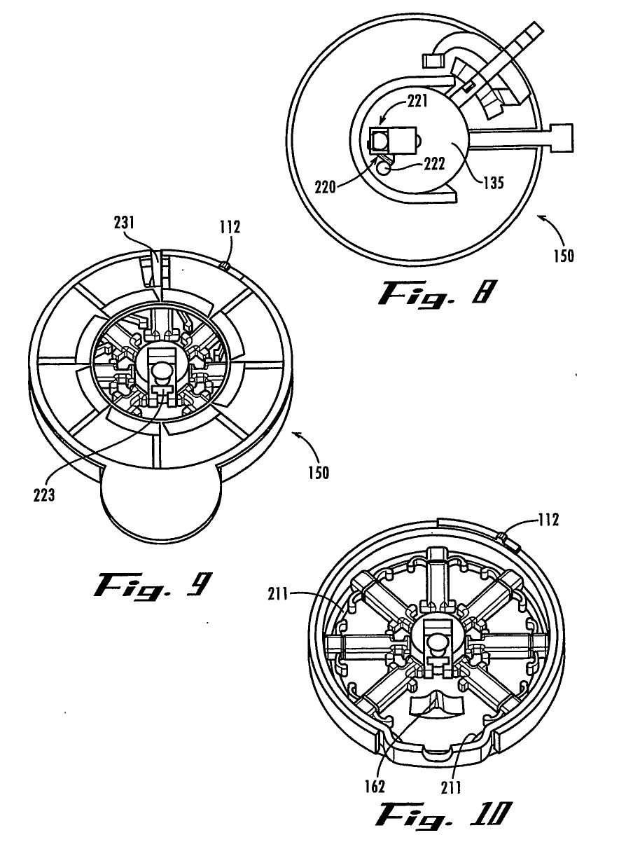

As shown in FIG. 7, after firing, the lancing device is rotationally advanced to recap the used lancet and bring another lancet into the active position. As the used lancet 171 moves from the firing position, the lancet moves transversely down along the bell-shaped cam 162 back into the storage plane, and the T-shaped end of the lancet body is released from the piston 139. The cap 183 is pushed back onto the used lancet under the influence of interplay of the cam track follower 191 on the lancet cap with the cam track 211. The movement back into the storage plane can be imparted under the influence of a spring or interacting surface features.

As shown in FIG. 8, a cam-actuated cocking mechanism is preferably provided to cock the drive mechanism of the lancing device. The cocking mechanism preferably comprises a raised flange or recessed track defining a cam path 220 located on the piston 221 and a post or pin 222 projecting from the arming lever 135. As the arming lever 135 is rotated, the pin 222 engages and passes along the cam path 220 to cock the piston 221 and energize the drive spring.

As depicted in FIG. 9, during advancement of the cassette 151, a pin 231 on the cassette 151 preferably follows in a slot in the in the housing. The slot for the pin 231 terminates at a position when the last lancet is in position for firing, so that the cassette 150 cannot be advanced any further, thereby preventing the reuse of the lancets in the cassette. Also, as previously described, the piston 221 captures the lancet as is moves out of the storage plane of the lancets and into the firing plane. The piston 221 has a T-shaped opening 223 for securely capturing and engaging the correspondingly shaped end of the lancet. Advantageously, the lancet is vertically captured in the piston with a positive engagement, rather than being laterally positioned within the piston. Although in the depicted embodiment the lancet engages the piston in a T-shaped arrangement, other arrangements, such as a ball-and-socket coupling, are within the scope of the invention.

With reference to FIGS. 9 and 10, a detent or projection 112 is preferably provided in or on the arming lever slot and must be overcome to provide a uniform torque/speed for each user, thereby minimizing speed variations and helping to prevent unintentional needle bending as the cap is removed. FIG. 10 also more clearly shows the cam track 211 for driving the caps as they are removed and replaced as the lancets move into and out of the firing position.

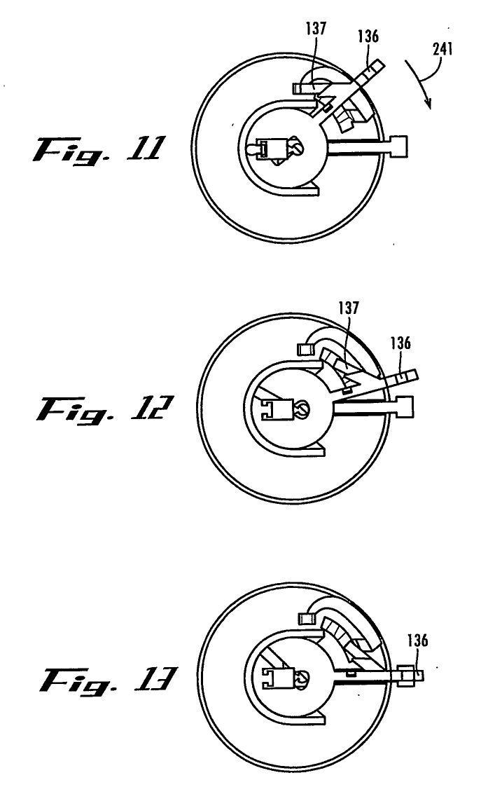

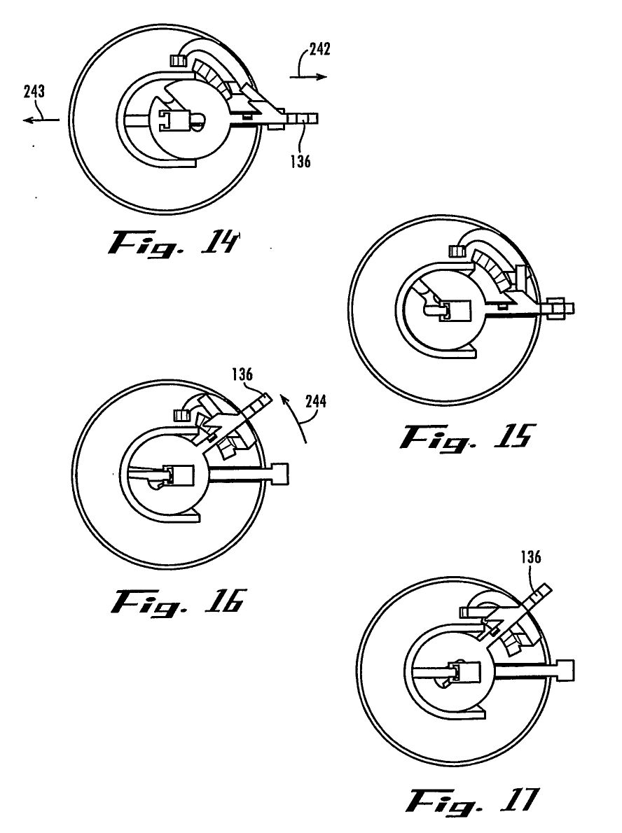

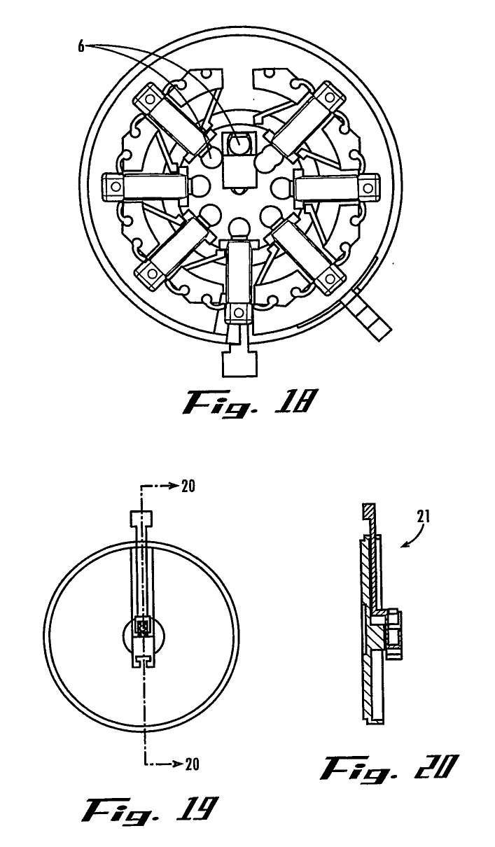

FIGS. 11-17 show the sequence of operation of the advancement mechanism for moving the cassette to bring each lancet sequentially into and through the active position, and the cocking mechanism for arming the drive mechanism for propelling the lancet. As shown in FIG. 11, the arming lever 136 is rotated clockwise in the direction indicated by direction arrow 241 to advance a lancet into the active position. As this happens, the pivotal pawl 137 is pulled along, in engagement with cooperating teeth on the cassette, to advance the cassette in the clockwise direction. In this way, the cassette is rotationally advanced relative to the housing of the lancing device. When the arming lever 136 reaches the position shown in FIG. 13, it has fully advanced the cassette to bring a new lancet into the firing position. The lancet is uncapped and moved from the storage plane into the firing plane, as described above. The drive mechanism is then cocked, by pulling the arming lever laterally in the direction of direction arrow 242 as shown in FIG. 14. In a simplified embodiment of the invention, the arming lever is then simply released, driving the piston forwardly in the direction of direction arrow 243 to lance the subject's skin at the sampling site. Alternatively, the arming lever 136 is released into the position shown in FIG. 15, and a separate trigger is actuated to fire the device. The arming lever 135 is released to return to its starting position, rotating back counterclockwise in the direction of direction arrow 244 until it reaches position of FIG. 17.

FIG. 18 shows an alternative embodiment of the invention in which the T-shaped ends of the lancets are replaced with balls and the T-shaped opening formed in the piston is replaced with a socket. Thus, as shown in this figure, a ball and socket arrangement replaces the T-shaped arrangement of the previous embodiment.

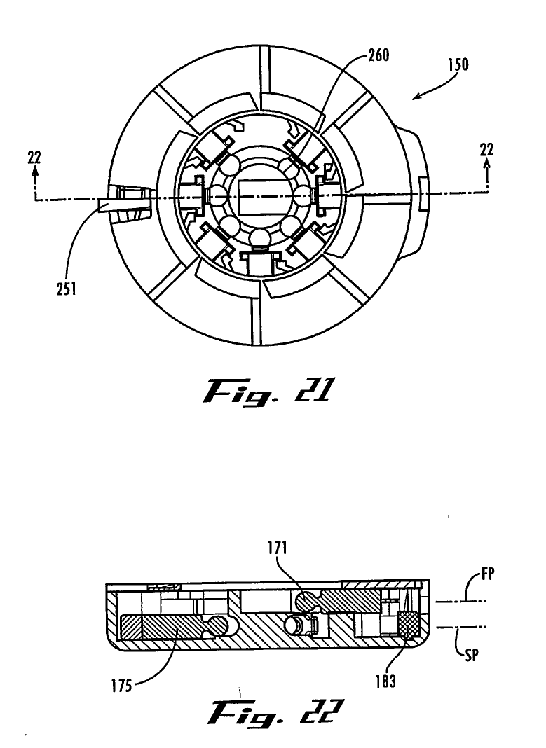

FIG. 19 is a bottom view of a portion of a lancing device 100, according to a modified form of the invention; and FIG. 20 is a side sectional view of the device of FIG. 19, taken along view lines A-A. In this embodiment, the drive spring is energized from the back, rather than pulling the piston back to load the back of the springs. In this arrangement, pushing the activation button cocks and fires the device all in one motion.

FIG. 21 is a top view of a portion of the sampling device of FIG. 18, and FIG. 22 is a side sectional view of the device of FIG. 21, taken along view lines A-A. As shown herein, a cantilever 251 on the cassette 150 drops into position when the last lancet is in the firing position to prevent further advancement of the cassette 150. As indicated generally at 260, after use, the lancets are returned to the storage plane, but are offset from their original positions so that they can not be aligned for engagement with the piston a second time, and therefore cannot be reused. Also, as seen in FIG. 22, the firing plane FP is common to both the lancet to be fired and the drive piston, while the storage plane SP is transversely offset therefrom. As shown in this figure, the cap 183 of the active lancet is held in the storage plane SP, while its associated lancet 171 is in the firing plane FP for firing. Meanwhile, the other lancets, such as lancet 175, are held in the storage plane SP.

As described herein, it will be recognized that the lancing device and lancet cartridge of the present invention provide a number of advantages over previously known devices. To assist in lancet retraction, a cantilever is provided on the cartridge body or cassette body, rather than locating it on the lancet. Lancets other than the active lancet are stored in a plane that is offset from the firing plane, and when a lancet is advanced into the active position for firing, it is pushed to the firing plane to align with and be engaged by the drive piston for firing to lance the subject's skin. After firing, the lancet is moved out of the firing plane and is returned to the storage plane. The cassette is installed into the housing from above and the lancets engage positively with the piston or plunger. The lancet is preferably vertically captured in the piston as it is moved transversely into the firing plane with a positive engagement, rather than being laterally positioned within the piston upon rotational advancement of the cartridge. The lancet preferably engages the piston by means of a T-shaped coupling arrangement or a ball-and-socket arrangement.

The lancet cap is preferably removed from the lancet prior to the lancet being advanced into the firing position. Extensible cantilevered arms preferably extend laterally from each side of the lancet cap to help maintain the position of the caps during uncapping and recapping. Rather than keeping the unfired lancets in the firing plane and moving them out of the firing plane after firing, the lancets are held in a storage plane transversely displaced from the firing plane and then moved into the firing plane for use. The lancet moving into the active position is held relatively stationary in the radial or axial direction (i.e., the direction in which the axis of the lancet needle lies) while the lancet cap is removed. In other words, the cap is pulled axially away from the lancet rather than the lancet being pulled away from the cap. The endcap is removed from the active lancet while the lancet and the cap together are rotationally advanced with the carrier, but relative to that rotational movement, the lancet preferably does not move in the axial direction while the cap is displaced radially outwardly away from the lancet.

The lancet cap cantilevers are preferably coupled to guide blocks of the cassette to retain the cap at all times and to help position the cap as it is displaced off of the lancet and replaced onto the lancet. The lancet cap guide or cam track is preferably a separate part of the cassette, rather than being a feature of the cap itself. The lancet is preferably retained and guided securely by the piston rather than being guided by the cartridge or cassette. During the firing of a lancet, all guidance is preferably provided by the piston guidance, rather than by the cassette.

The central post in the cassette preferably prevents other lancets from interfering with the piston before or after the firing of a lancet. Fingers or springs positioned in the housing base preferably push upwardly on the cassette to position the cassette and to ensure full engagement of the arming lever with the cassette. A breakaway feature is preferably provided on the cassette to assist in the insertion of the cassette in the device. When the breakaway feature has been removed, it is difficult to properly insert a cassette, which discourages the improper reuse of cassettes. Spring fingers in the base of the housing preferably also push the cassette upwardly when the housing is open, thereby assisting in the removal of the used cassette from the housing.

To arm the lancing device, the arming lever is preferably rotated within a slot to advance the cassette and to engage the lancet and lancet holder. The arming lever is then preferably pulled back to cock the piston. Alternatively, a cam path is provided to cock the lancing device, which cam path is preferably located on the piston and a cooperating post or pin is provided on the arming lever. As the arming lever rotates, the pin passes over the cam path and cocks the piston. Alternatively, the drive spring is loaded from the back, rather than pulling the piston back to load the back of the springs, whereby pushing the activation button cocks and fires the device in a single motion.

During advancement of the cassette, a pin on the cassette preferably follows in a slot in the in the housing. The slot for the pin preferably terminates at a position when the last lancet is in position for firing. The cassette preferably cannot be advanced any further, thereby preventing the reuse of lancets. After firing, as the next lancet is moved into the firing position, the used lancet is preferably returned to the storage position and is recapped to prevent the reuse of the lancet and to prevent accidental needle sticks upon removal of the used cassette. A cantilever on the cassette preferably drops into position when the last lancet enters the firing position, to prevent further advancement of the cassette. The arming lever preferably does not have a ledge to engage with when the last lancet is in the firing position, so the arming lever cannot advance the cassette any further. After use, the lancets are preferably returned to the storage plane, but are axially offset from their original positions so that they will not be able to be aligned with the piston a second time and therefore cannot be reused.

The lancing device preferably includes an access cover that can be rotated out of the way to provide access to a sensor disk for use and then rotated back to close the sensor cavity. Chemical leads are optionally provided to connect a blood collection area with an analyte processing device, or, in an alternative embodiment, chemical leads can provide positive connections between a blood receptacle and electrically conductive leads.

The lancet cap preferably blocks the sharp lancet tip to prevent the tip from extending outside of lancing device before and after firing. A detent is preferably provided in the arming lever slot and must be overcome to provide a uniform torque/speed for each user, thereby minimizing speed variations and helping to prevent unintentional needle bending as the cap is removed. An interlock system is preferably provided in the arming lever to prevent the arming lever from sliding through its slot once the device is cocked. Once the lancing device has been fired, the arming lever is preferably free to slide once again. The arming lever preferably has a cantilever portion that slides through a path in the housing as the arming lever slides through its slot. When the arming lever is advancing the cassette, the cantilever preferably passes through a path with several ledges or teeth for engagement so the arming lever will not return to the starting position before it slides through its full stroke. The cantilever is preferably disengaged from the ledges or teeth when the arming lever is pulled back to cock the lancing device and the arming lever is free to be returned to the start position.

While the invention has been described with reference to preferred and example embodiments, it will be understood by those skilled in the art that a variety of modifications, additions and deletions are within the scope of the invention, as defined by the following claims.

What is claimed is:

1. A lancing device comprising a plurality of lancets positioned in a planar array, said planar array defining a storage plane, wherein an active lancet selected from the plurality of lancets is transversely displaced out of the storage plane into a firing plane that is generally parallel to the storage plane.

2. The lancing device of claim 1, wherein the plurality of lancets are positioned in a circular pattern within the planar array, with sharp tips of the lancets oriented radially outward.

3. The lancing device of claim 1, wherein the active lancet is transversely displaced out of the storage plane into a firing plane upon rotational advancement of the plurality of lancets.

4. The lancing device of claim 3, further comprising a cam surface for transversely displacing the active lancet into the firing plane.

5. The lancing device of claim 1, wherein the active lancet moves into engagement with a drive mechanism as it is transversely displaced out of the storage plane into a firing plane.

6. The lancing device of claim 5, wherein each lancet comprises a T-shaped end for engagement with a slotted coupling on a piston portion of the drive mechanism.

7. The lancing device of claim 5, wherein each lancet comprises a ball-shaped end for engagement with a socket coupling on a piston portion of the drive mechanism.

8. The lancing device of claim 1, wherein each lancet comprises a removable endcap, and wherein the endcap is separated from the active lancet before the active lancet is transversely displaced out of the storage plane into the firing plane.

9. The lancing device of claim 8, wherein each lancet comprises a follower in engagement with a cooperating cam surface for separating the endcap from the active lancet.

10. The lancing device of claim 8, wherein the active lancet is constrained against axial movement as the endcap is separated therefrom.

11. The lancing device of claim 8, wherein the removable endcap of each lancet comprises at least one extensible arm extending laterally therefrom, for guiding the position of the endcap upon separation from the active lancet.

12. The lancing device of claim 1, wherein the active lancet is guided by a drive mechanism along a lancing path in the firing plane, between a retracted position fully within a housing of the lancing device and an extended position wherein at least a sharp tip portion of the active lancet extends outwardly from the housing.

13. The lancing device of claim 12, wherein the housing comprises a base and a cover, said cover defining an opening, and further comprising a movable door for selectively covering and uncovering the opening.

14. The lancing device of claim 1, wherein the plurality of lancets are contained within a disposable cassette that is removably installed within a reusable housing of the lancing device.

15. The lancing device of claim 14, wherein the housing comprises a biasing member for biasing the disposable cassette out of the housing to assist in removal.

16. The lancing device of claim 14, wherein the cassette initially comprises a breakaway tab to assist in installing the disposable cassette in the housing.

17. The lancing device of claim 14, further comprising an advancing mechanism comprising a rotational arming lever having a pawl for cooperative engagement with teeth of the disposable cassette for moving lancets sequentially into an active position.

18. The lancing device of claim 17, wherein the pawl is pivotally coupled to the rotational arming lever.

19. The lancing device of claim 17, wherein the rotational arming lever comprises a cam path for cooperative engagement with a follower portion of a drive mechanism, and wherein rotational actuation of the arming lever energizes the drive mechanism.

20. The lancing device of claim 1, wherein a used lancet is returned from the firing plane to the storage plane.

21. The lancing device of claim 20, wherein the used lancet is returned to the storage plane in an axially-offset position to prevent reuse.

22. A lancing device comprising a plurality of lancets positioned in a planar array, said planar array defining a storage plane, wherein an active lancet selected from the plurality of lancets is transversely displaced out of the storage plane into a firing plane that is generally parallel to the storage plane, and wherein each lancet comprises a removable endcap which is separated from the active lancet before the active lancet is transversely displaced out of the storage plane into the firing plane, the active lancet being constrained against axial movement as the endcap is separated therefrom.

23. A lancing device comprising at least one lancet having a sharp tip and a protective endcap covering said sharp tip, said lancing device further comprising a decapping mechanism for removing the endcap from the lancet without axial displacement of the lancet.

24. The lancing device of claim 23, wherein the decapping mechanism comprises a follower on said endcap, said follower being engaged with a cooperating cam track for radial displacement of the endcap away from the lancet as the lancet is laterally advanced.

25. The lancing device of claim 23, wherein the protective endcap comprises a pair of extensible arms extending laterally from opposed sides thereof, said arms guiding the position of the endcap as it is removed from the lancet.

26. A lancing device comprising a lancet movable between a retracted position and a lancing position, said lancet comprising a lancet body with a wing projecting outwardly therefrom, and wherein said lancing device further comprises a retraction spring separate from the lancet, said retraction spring contacting the outwardly projecting wing when the lancet is in the lancing position to bias the lancet toward the retracted position.

27. A lancing device comprising a plurality of lancets, an advancing mechanism for advancing each of the plurality of lancets sequentially into an active position, and a drive mechanism for propelling an active lancet selected from the plurality of lancets from a retracted position to a lancing position, wherein the advancing mechanism comprises an arming lever that is rotationally actuated to advance the lancets, and linearly retracted to energize the drive mechanism.

28. The lancing device of claim 26, wherein the arming lever comprises a cam path engaging a follower portion of the drive mechanism, whereby rotational actuation of the arming lever advances the lancets and energizes the drive mechanism.

29. A lancing device comprising a reusable housing and a cartridge containing a plurality of lancets, the cartridge being removably installed within the housing, said lancing device further comprising a pin extending from the cartridge and movable through a slot in the housing as the cartridge is advanced, wherein the slot has an end preventing further movement of the pin and thereby blocking further advancement of the cartridge to prevent reuse of a lancet.

30. The lancing device of claim 29, wherein the means for preventing reuse of a lancet comprises a cantilevered member on the cartridge, which drops into engagement with the housing to block advancement of the cartridge.

31. The lancing device of claim 29, wherein the means for preventing reuse of a lancet comprises a plurality of ledges in a spaced array on the cartridge for engagement with an advancing mechanism, and wherein the spaced array extends only partially along the cartridge so that the advancing mechanism cannot advance the cartridge beyond an end of the array.

32. The lancing device of claim 29, wherein the means for preventing reuse of a lancet comprises a positioning mechanism for returning used lancets from a firing position to an axially-offset storage position.

33. A cassette for removable insertion within a lancing device, said cassette comprising a plurality of lancets positioned in a planar array, said planar array defining a storage plane, and wherein an active lancet selected from the plurality of lancets is transversely displaceable out of the storage plane into a firing plane within the cassette.

34. The cassette of claim 33, comprising an outer shell containing the plurality of lancets.

35. The cassette of claim 34, wherein each lancet comprises a removable endcap with a follower that rides along a cam track in the outer shell.

36. The cassette of claim 33, further comprising a cam surface projecting transversely inward from the outer shell for displacing the active lancet into the firing plane as the active lancet is laterally advanced.

37. The cassette of claim 34, wherein a plurality of guide blocks are provided within the outer shell, and wherein each lancet comprises at least one extensible lateral arm connected to one of said guide blocks.

38. The cassette of claim 37, wherein the plurality of lancets are sequentially movable through an active position wherein the active lancet traverses a lancing stroke between a retracted position within the outer shell and an extended position wherein at least a sharp tip portion thereof projects out of the outer shell, and wherein a retraction spring extends from each guide block for contact with a cooperating portion of one of the plurality of lancets when in the extended position.

39. The cassette of claim 33, further comprising a breakaway handle portion.

40. The cassette of claim 33, wherein the plurality of lancets are positioned in a circular pattern within the planar array, with sharp tips of the lancets oriented radially outward.

41. A cassette for removable insertion within a lancing device, said cassette comprising a lancet having a lancet body, a sharp tip projecting from the lancet body, and a protective endcap separably affixed over the sharp tip, said protective endcap comprising a follower in engagement with a cam track, said cam track defining a path for axially displacing the protective endcap from the lancet body as the lancet is laterally advanced within the cassette.

42. The cassette of claim 41, wherein the plurality of lancets are positioned in a circular pattern within the planar array, with sharp tips of the lancets oriented radially outward, and wherein the cam track is generally circular, with an outwardly-directed eccentric portion where the protective endcap is displaced from the lancet body.
